(19) United States
(12) Patent Application Publication
(10) Pub. No.: US 2022/0196682 A1
(43) Pub. Date: Jun. 23, 2022

(54) CATECHOLAMINE CONCENTRATIONS IN A BLOOD SAMPLE AND ARTERIAL HYPERTENSION STATUS IN A SUBJECT FOR A DIFFERENTIAL DIAGNOSIS OF NEURODEGENERATIVE DISEASES INCLUDING ALZHEIMER'S DISEASE

(71) Applicant: SAS ALZOHIS, Paris (FR)

(72) Inventors: Romain VERPILLOT, Paris (FR); Hervé THIRIEZ, Versailles (FR); Rudy BEAUJEAN, Paris (FR)

(73) Assignee: SAS ALZOHIS, Paris (FR)

(21) Appl. No.: 17/594,614

(22) PCT Filed: Apr. 17, 2020
(86) PCT No.: PCT/EP2020/060902

§ 371 Date: Oct. 25, 2021

(30) Foreign Application Priority Data
Apr. 25, 2019 (LU) LU101191

(51) Int. Cl. G01N 33/68; G01N 33/94

(52) U.S. Cl. CPC G01N 33/6896; G01N 33/9433; G01N 33/9413

(57) ABSTRACT

The invention has for object an in vitro method for performing a differential diagnosis of neurodegenerative diseases selected from early stage of Alzheimer's disease, advanced stage of Alzheimer's disease, mental depression, dementia with Lewy bodies, frontotemporal dementia, and/or vascular dementia; said method being performed in a subject presenting signs of dementia and comprising the steps of (a) determining at least five criteria of said subject, said at least five criteria comprising the age of said subject, a score of said subject to a questionnaire adapted for screening cognitive function, a dopamine, adrenaline and noradrenaline concentration in a blood sample of said subject, (b) comparing said noradrenaline concentration to a predetermined threshold, (c) calculating one global note in relation with each neurodegenerative disease and (d) determining whether said subject suffers from one or more neurodegenerative diseases.

### RELATED APPLICATIONS

This application is a national phase application of and claims priority under 35 U.S.C. § 371 of PCT Patent Application Serial No. PCT/EP2020/060902 filed on Apr. 17, 2020 and titled CATECHOLAMINE CONCENTRATIONS INA BLOOD SAMPLE AND ARTERIAL HYPERTENSION STATUS IN A SUBJECT FOR A DIFFERENTIAL DIAGNOSIS OF NEURODEGENERATIVE DISEASES INCLUDING ALZHEIMER'S DISEASE. The content of this application is incorporated herein by reference.

### FIELD OF THE INVENTION

The present invention relates to a method for performing a differential diagnosis of neurodegenerative diseases in a subject.

### BACKGROUND OF THE INVENTION

Continuous technological development in healthcare have saved countless lives and improved the quality of live for even more. In the case of a subject suffering from neurodegenerative disease(s), not only the patient is affected but also its family or close acquaintance. Currently, the treatment of neurodegenerative only tries to improve the living conditions of the patients but cannot completely cure the patient. However, it is important to give to one patient the good treatment to alleviate its pain in the best way as possible. More importantly, sooner the diagnosis can be performed, better the treatment can be adjusted. The whole task is not straightforward, since sometimes the subject may also suffer from mixed dementia. Such a patient shows symptoms of two or more neurodegenerative diseases in the same time and it is very difficult for a clinician to determine the adequate treatment. For instance, a patient can be diagnosed of suffering from Alzheimer's disease (AD) and vascular dementia (VD), or from AD and dementia with Lewy bodies (DLB). It also happened in the case of a patient suffering from mixed dementia that one dementia is predominant over the other, which needs further adjustment of the medical treatment.

In WO 92/13273, a method for diagnosing disorders in living organisms is described. In this method, the fluid samples from normal and abnormal individuals are analysed to generate patterns representative of molecular constituents of said samples. A database of frequency distribution patterns of constituents of samples from organisms having known categories of disorders and controls is thus created, and the unknown sample analysis is compared for conformity to said frequency distribution patterns. This allows to evaluate both treatment protocols and new pharmaceuticals.

With regard to the neurodegenerative diseases, and Alzheimer's disease (AD) in particular, strong evidences for catecholaminergic systems alterations have been highlighted. EP 2 756 311 relates to an in vitro method for diagnosing Alzheimer's disease in a subject presenting signs of dementia which is based on the determination of the level of dopamine, adrenaline and noradrenaline in a sample of biological fluid from the patient. In this case, only a patient suffering from AD can be diagnosed, leaving the rest of the group of patients unaware of their disease.

Identification of neurodegenerative disease constructed on medical imaging and analysis of cerebrospinal fluid (CSF) is quite expensive and sometimes complicated to implement to elderly patient.

The invention has for objective to provide a method which overcomes the inherent difficulties of obtaining an accurate diagnosis of one subject selected among a group of subjects suffering from a panel of neurodegenerative diseases. In particular, the invention has for objective to provide a method which is able to accurately diagnose one subject selected among a group of subjects suffering from Alzheimer's disease, mental depression, dementia with Lewy bodies, frontotemporal dementia, vascular dementia, or mixed dementia.

### SUMMARY OF THE INVENTION

According to a first aspect, the invention has for object an in vitro method for performing a differential diagnosis of neurodegenerative diseases selected from early stage of Alzheimer's disease (AD1), advanced stage of Alzheimer's disease (AD2), mental depression (PSY), dementia with Lewy bodies (DLB), frontotemporal dementia (FTD), and/or vascular dementia (VD); said method being performed in a subject presenting signs of dementia;

said method comprising the steps of:
a) determining at least five criteria of said subject, said at least five criteria comprising the age of said subject, a score of said subject to a questionnaire adapted for screening cognitive function, a dopamine concentration [D] in a blood sample of said subject, an adrenaline concentration [A] in said blood sample of said subject and a noradrenaline concentration [NA] in said blood sample of said subject,
c) calculating one global note per each neurodegenerative disease according to the following sub-steps:
   i) determining for one neurodegenerative disease one parameter V_{disease}^{criterion} per criterion, said parameter combining one criterion selected from the five criteria determined in step (a) with reference values of said one criterion in function of said one neurodegenerative disease,
   ii) summing each parameter related to said one neurodegenerative disease obtained in sub-step (i) so as to obtain one global note in relation with said one neurodegenerative disease;
   iii) repeating sub-steps (i) and (ii) for each neurodegenerative disease,
d) determining, based on each global note per neurodegenerative disease calculated in steps (c), whether said subject suffers from one or more neurodegenerative diseases selected from early stage Alzheimer's disease (AD1), advanced stage of Alzheimer's disease (AD2), mental depression (PSY), dementia with Lewy bodies (DLB), frontotemporal dementia (FTD), and/or vascular dementia (VD);
   said method being remarkable in that it further comprises:
a step (b) performed after step (a); wherein step (b) comprises the step of comparing said noradrenaline concentration [NA] to a predetermined threshold, and if said noradrenaline concentration [NA] is lower or equal to said predetermined threshold, then determining the arterial hypertension status of said subject,
and in that step (c) further comprises, if said noradrenaline concentration [NA] is lower or equal to said predetermined threshold, the step of calculating the global note in relation with advanced stage of Alzheimer's disease (AD2) by adding a parameter V\hat{ }HT in relation with the arterial hypertension status of said subject.

Surprisingly, the inventors have found that it is possible to provide a precise diagnosis of neurodegenerative diseases by the analysis of at least five criteria of a subject, wherein the five criteria are combined together with reference values characteristic of each of the following neurodegenerative diseases selected from early stage of Alzheimer's disease (AD1), advanced stage of Alzheimer's disease (AD2), mental depression (PSY), dementia with Lewy bodies (DLB), frontotemporal dementia (FTD) and/or vascular dementia (VD). The unique combination of the five criteria and the additional criterion related to the arterial hypertension status followed by the determination of a global note allows for providing a method of diagnosis that can determine whether the subject suffers from AD, mental depression, DLB, FTD, VD and/or mixed dementia.

It is further notable that the diagnosis is not invasive and is performed in vitro on a blood sample easily collected (by comparison with a lumbar puncture for example) from the subjects. Moreover, this allows for the repeatability of the diagnosis and thus for statistical confirmation of the diagnosis.

The accuracy of the differential diagnosis is such that it can also diagnose between two distinct stages of Alzheimer's disease, namely an early stage of Alzheimer's disease and an advanced stage of Alzheimer's disease.

Such differential diagnosis allows the categorization of the subject according to her/his disease and this is an opportunity for such a person to receive adequate medical treatments. Moreover, the diagnosis allows for identifying subjects who are suffering from mixed dementia, which is not easy since such type of subjects can show symptoms that are relevant for two or more neurodegenerative diseases.

The following features of the in vitro method for performing a differential diagnosis of neurodegenerative disease in one subject are advantageously used to better define the present invention.
Said subject is in homeostatic imbalance.
The predetermined threshold of step (b) is a noradrenaline concentration [NA] comprised between 2900 pmol/l and 3100 pmol/l, preferentially comprised between 2950 pmol/l and 3050 pmol/l, more preferentially comprised between 2975 pmol/l and 3025 pmol/l, even more preferentially is a noradrenaline concentration [NA] of at least 3000 pmol/l.
In one instance, the predetermined threshold is a noradrenaline concentration [NA] of 3023 pmol/l.
Said steps (b), (c) and (d) are carried out by a computer.
The questionnaire adapted for screening cognitive function is a mini-mental state examination (MMSE) questionnaire.
Said parameter V_{disease}^{criterion} per criterion in function of each neurodegenerative disease is determined in sub-step (i) according to the following mathematical equation (1):

$${V}_{d i s e a s e}^{c r i t e r i o n} = 1 + K \frac{V A {L}^{criterion} - {MINI}_{disease}^{criterion}}{{MAXI}_{disease}^{criterion} - {MINI}_{disease}^{criterion}} \qquad ( 1 )$$

wherein VAL^{criterion} is the measured value of one of the five criteria determined in step (a),
wherein MINI_{disease}^{criterion} is the minimum reference value with respect to each neurodegenerative disease in function of each criterion,
wherein MAXI_{disease}^{criterion} is the maximum reference value with respect to each neurodegenerative disease in function of each criterion, and
wherein K is a mathematical parameter, K being comprised between 0.01 and 1.00, preferentially between 0.02 and 0.99, more preferentially between 0.05 and 0.95, even more preferentially between 0.10 and 0.90.
The proviso that if VAL^{criterion}<MINI_{disease}^{criterion} or if if VAL^{criterion}>MAXI_{disease}^{criterion} then V_{disease}^{criterion}=0.
Said in vitro method for performing a differential diagnosis of neurodegenerative disease in one subject further comprises the step of attributing a weighting factor to each parameter V_{disease}^{criterion}. With preference, said weighting factor is comprised between 1.0 and 10.0 with respect to one or more parameters selected from the parameter in relation with the age of said subject, the parameter in relation with the dopamine concentration [D], the parameter in relation with the adrenaline concentration [A] and the parameter in relation with the noradrenaline concentration [NA]; preferentially the weighting factor is 5.0; and/or, said weighting factor is comprised between 0.1 and 3.0 with respect to the parameter in relation with the score to a questionnaire adapted for screening cognitive function of said subject; preferentially the weighting factor with respect to the parameter in relation with the score to a questionnaire adapted for screening cognitive function of said subject is 2.0.
Said parameter V^{HT} in relation with the arterial hypertension status of said subject is function of the age of said subject, and is comprised between 0.0 and 11.0; preferentially between 0.1 and 5.0. In one instance, the said parameter V^{HT} in relation with the arterial hypertension status of said subject is 3.0.
Said parameter V^{HT} in relation with the arterial hypertension status of said subject is determined according to the following mathematical equation (2)

V^{HT}=α(age)+β (2)
wherein α is a variable in function of the age of said subject; with preference α is a value comprised between 0.0 and 10.0.
wherein β is a variable in function of the seriousness of the arterial hypertension status; with preference β is a value comprised between 0.0 and 1.0.
Said in vitro method for performing a differential diagnosis of neurodegenerative disease in one subject comprises the step of applying a correction factor 6 to said reference values, said correction factor δ being comprised between 0.50 and 1.50, preferentially between 0.60 and 1.40.
Said blood sample of said subject is one plasma sample of said subject.
Said noradrenaline, dopamine and adrenaline concentrations are determined with an HPLC equipped with an electrochemical detector.
Said step (d) of determining whether said subject suffers from one or more neurodegenerative diseases selected from early stage Alzheimer's disease (AD1), advanced stage of Alzheimer's disease (AD2), mental depression (PSY), dementia with Lewy bodies (DLB), frontotemporal dementia (FTD), and/or vascular dementia (VD) is performed by comparing the global notes obtained in relation with each of neurodegenerative diseases.
Said step (d) of determining whether said subject suffers from one or more neurodegenerative diseases selected from early stage Alzheimer's disease (AD1), advanced stage of Alzheimer's disease (AD2), mental depression (PSY), dementia with Lewy bodies (DLB), frontotemporal dementia (FTD), and/or vascular dementia (VD) is equivalent to a step of determining whether said subject suffers from one neurodegenerative disease selected from early stage Alzheimer's disease (AD1), advanced stage of Alzheimer's disease (AD2), mental depression (PSY), dementia with Lewy bodies (DLB), frontotemporal dementia (FTD), or vascular dementia (VD) or whether said subject suffers from mixed dementia.
Said step (a) further comprises the determination of one or more of the following criteria, said criteria being the sex of said subject or a presence of an APOE□4 allele in the genotype of said subject.

In a preferred embodiment, the reference values used in step (c) in relation with early stage of Alzheimer's disease (AD1) are as followed:
with respect to the age of said subject, the minimum reference value is at least 50 years, preferably at least 51 years, more preferably is 52.92; and/or the maximum reference value is at most 90 years; preferably at most 89 years, more preferably is 87.72 years;
with respect to the score of said subject to a questionnaire adapted for screening cognitive function, the minimum reference value is at least 14, preferably is 15; and/or the maximum reference value is at most 29, preferably is 28;
with respect to the dopamine concentration [D], the minimum reference value is at least 90 pmol/l, preferably at least 100 pmol/l, more preferably is 110.74 pmol/l; and/or the maximum reference value is at most 1000 pmol/l, preferably at most 900 pmol/l, more preferably is 878.22 pmol/l;
with respect to the adrenaline concentration [A] the minimum reference value is at least 40 pmol/l, preferably at least 50 pmol/l, more preferably is 65.66 pmol/l; and/or the maximum reference value is at most 2000 pmol/l, preferably at most 1900 pmol/l, more preferably is 1720.74 pmol/l;
with respect to the noradrenaline concentration [NA] the minimum reference value is at least 2900 pmol/l, preferably at least 2950 pmol/l, more preferably is 2962.54 pmol/l; and/or the maximum reference value is at most 9000 pmol/l, preferably at most 8800 pmol/l, more preferably is 8681.22 pmol/l.

In a preferred embodiment, the reference values used in step (c) in relation with advanced stage of Alzheimer's disease (AD2) are as followed:
with respect to the age of said subject, the minimum reference value is at least 45 years, preferably at least 50 years, more preferably is 51.94; and/or the maximum reference value is at most 95 years, preferably at most 90 years, more preferably is 89.76 years;
with respect to the score of said subject to a questionnaire adapted for screening cognitive function, the minimum reference value is at least 3, preferably is 4; and/or the maximum reference value is at most 30, preferably is 29;
with respect to the dopamine concentration [D], the minimum reference value is at least 140 pmol/l, preferably at least 150 pmol/l, more preferably is 152.88 pmol/l; and/or the maximum reference value is at most 1000 pmol/l, preferably at most 800 pmol/l, more preferably is 655.86 pmol/l;
with respect to the adrenaline concentration [A] the minimum reference value is at least 80 pmol/l, preferably at least 100 pmol/l, more preferably is 143.08 pmol/l; and/or the maximum reference value is at most 1500 pmol/l, preferably at most 1400 pmol/l, more preferably is 1371.09 pmol/l;
with respect to the noradrenaline concentration [NA] the minimum reference value is at least 750 pmol/l, preferably at least 850 pmol/l, more preferably is 1023.12 pmol/l; and/or the maximum reference value is at most 3000 pmol/l, preferably at most 2900 pmol/l, more preferably is 2758.08 pmol/l.

In a preferred embodiment, the reference values used in step (c) in relation with mental depression (PSY) are as followed:
with respect to the age of said subject, the minimum reference value is at least 35 years, preferably at least 37 years, more preferably is 40.18; and/or the maximum reference value is at most 80 years, preferably at most 75 years, more preferably is 74.46 years;
with respect to the score of said subject to a questionnaire adapted for screening cognitive function, the minimum reference value is at least 21, preferably is 22; and/or the maximum reference value is at most 30, preferably is 29;
with respect to the dopamine concentration [D], the minimum reference value is at least 250 pmol/l, preferably at least 260 pmol/l, more preferably is 276.36 pmol/l; and/or the maximum reference value is at most 750 pmol/l, preferably at most 650 pmol/l, more preferably is 519.18 pmol/l;
with respect to the adrenaline concentration [A] the minimum reference value is at least 180 pmol/l, preferably at least 185 pmol/l, more preferably is 191.10 pmol/l; and/or the maximum reference value is at most 450 pmol/l, preferably at most 425 pmol/l, more preferably is 406.98 pmol/l;
with respect to the noradrenaline concentration [NA] the minimum reference value is at least 1000 pmol/l, preferably at least 1200 pmol/l, more preferably is 1533.70 pmol/l; and/or the maximum reference value is at most 4000 pmol/l, preferably at most 3800 pmol/l, more preferably is 3691.38 pmol/l.

In a preferred embodiment, the reference values used in step (c) in relation with dementia with Lewy bodies (DLB) are as followed:
with respect to the age of said subject, the minimum reference value is at least 55 years, preferably is at least 60 years, more preferably is 61.74; and/or the maximum reference value is at most 85 years, preferably at most 83 years, more preferably is 81.6 years;
with respect to the score of said subject to a questionnaire adapted for screening cognitive function, the minimum reference value is at least 7, preferably is 8; and/or the maximum reference value is at most 25, preferably is 24;
with respect to the dopamine concentration [D], the minimum reference value is at least 150 pmol/l, preferably at least 175 pmol/l, more preferably is 203.84 pmol/l; and/or the maximum reference value is at most 400 pmol/l, preferably at most 390 pmol/l, more preferably is 375.36 pmol/l;
with respect to the adrenaline concentration [A] the minimum reference value is at least 80 pmol/l, preferably at least 90 pmol/l, more preferably is 103.88 pmol/l; and/or the maximum reference value is at most 700 pmol/l, at most 600 pmol/l, more preferably is 418.20 pmol/l;
with respect to the noradrenaline concentration [NA] the minimum reference value is at least 900 pmol/l, preferably at least 1000 pmol/l, more preferably is 1065.26 pmol/l; and/or the maximum reference value is at most 8000 pmol/l, preferably at most 7500 pmol/l, more preferably is 7216.50 pmol/l.

In a preferred embodiment, the reference values used in step (c) in relation with frontotemporal dementia (FTD) are as followed:
with respect to the age of said subject, the minimum reference value is at least 45 years, preferably at least 47 years, more preferably is 49; and/or the maximum reference value is at most 80 years, preferably at most 77 years, more preferably is 75.48 years;
with respect to the score of said subject to a questionnaire adapted for screening cognitive function, the minimum reference value is at least 0, preferably is 0; and/or the maximum reference value is at most 28, preferably is 27;
with respect to the dopamine concentration [D], the minimum reference value is at least 200 pmol/l, preferably at least 220 pmol/l, more preferably is 245.98 pmol/l; and/or the maximum reference value is at most 1100 pmol/l, preferably at most 1050 pmol/l, more preferably is 1002.66 pmol/l;
with respect to the adrenaline concentration [A] the minimum reference value is at least 100 pmol/l, preferably at least 110 pmol/l, more preferably is 123.48 pmol/l; and/or the maximum reference value is at most 1500 pmol/l, preferably at most 1400 pmol/l, more preferably is 1270.92 pmol/l;
with respect to the noradrenaline concentration [NA] the minimum reference value is at least 700 pmol/l, preferably at least 800 pmol/l, more preferably is 874.16 pmol/l; and/or the maximum reference value is at most 6000 pmol/l, preferably at most 5900 pmol/l, more preferably is 5743.62 pmol/l.

In a preferred embodiment, the reference values used in step (c) in relation with vascular dementia (VD) are as followed:
with respect to the age of said subject, the minimum reference value is at least 55 years, preferably at least 57 years, more preferably is 58.8; and/or the maximum reference value is at most 90 years, preferably at most 87 years, more preferably is 85.68 years;
with respect to the score of said subject to a questionnaire adapted for screening cognitive function, the minimum reference value is at least 20, preferably is 21; and/or the maximum reference value is at most 29, preferably is 28;
with respect to the dopamine concentration [D], the minimum reference value is at least 140 pmol/l, preferably at least 160 pmol/l, more preferably is 198.94 pmol/l; and/or the maximum reference value is at most 580 pmol/l, preferably at most 560 pmol/l, more preferably is 533.46 pmol/l;
with respect to the adrenaline concentration [A] the minimum reference value is at least 200 pmol/l, preferably at least 220 pmol/l, more preferably is 240.10 pmol/l; and/or the maximum reference value is at most 1800 pmol/l, preferably at most 1700 pmol/l, more preferably is 1525.92 pmol/l;
with respect to the noradrenaline concentration [NA] the minimum reference value is at least 1400 pmol/l, preferably at least 1450 pmol/l, more preferably is 1518.02 pmol/l; and/or the maximum reference value is at most 4000 pmol/l, preferably at most 3900 pmol/l, more preferably is 3705.66 pmol/l.

In a preferred embodiment, the step (c) further comprises calculating one global note in relation with neurological control (NC); with preference, the reference values used in step (c) in relation with neurological control (NC) are as followed:
with respect to the age of said subject, the minimum reference value is at least 35 years, preferably at least 37 years, more preferably is 40.18; and/or the maximum reference value is at most 90 years, preferably at most 87 years, more preferably is 84.66 years;
with respect to the score of said subject to a questionnaire adapted for screening cognitive function, the minimum reference value is at least 22, preferably is 23; and/or the maximum reference value is at most 30, preferably is 30;
with respect to the dopamine concentration [D], the minimum reference value is at least 180 pmol/l, preferably at least 200 pmol/l, more preferably is 207.76 pmol/l; and/or the maximum reference value is at most 1600 pmol/l, preferably at most 1500 pmol/l, more preferably is 1334.16 pmol/l;
with respect to the adrenaline concentration [A] the minimum reference value is at least 110 pmol/l, preferably at least 130 pmol/l, more preferably is 163.66 pmol/l;
and/or the maximum reference value is at most 2500 pmol/l, preferably at most 2400 pmol/l, more preferably is 2366.40 pmol/l;
with respect to the noradrenaline concentration [NA] the minimum reference value is at least 900 pmol/l, preferably at least 910 pmol/l, more preferably is 925.12 pmol/l; and/or the maximum reference value is at most 7000 pmol/l, preferably at most 6600 pmol/l, more preferably is 6331.14 pmol/l.

According to a second aspect, the invention has for object a data processing system comprising a processor configured to perform the method in accordance with the first aspect when said steps (b), (c) and (d) are carried out by a computer.

According to a third aspect, the invention has for object a computer program comprising instructions which, when the program is executed by a computer, cause the computer to carry out the method in accordance with the first aspect when said steps (b), (c) and (d) are carried out by the computer.

According to a fourth aspect, the invention has for object a computer-readable data carrier having stored thereon the computer program in accordance with the third aspect of the invention.

### BRIEF DESCRIPTION OF THE DRAWINGS

FIG. 1 schematically shows the concept of the differential diagnosis according to the invention.

FIG. 2 shows the histogram distribution of the four groups of subjects: AD1, AD2, FTD/DLB/VD and PSY/NC.

FIG. 3 shows the relative frequency distribution of plasma [NA] concentrations of AD (n=50) and non-AD (n=50) subjects.

FIG. 4 shows the Receiver Operating Characteristic (ROC) curve of plasma [NA] concentrations in AD versus non-AD subjects.

### DETAILED DESCRIPTION OF THE INVENTION

For the purpose of the invention, the following definitions are given:

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

The recitation of numerical ranges by endpoints includes all integer numbers and, where appropriate, fractions subsumed within that range (e.g. 1 to 5 can include 1, 2, 3, 4 when referring to, for example, a number of elements, and can also include 1.5, 2, 2.75 and 3.80, when referring to, for example, measurements). The recitation of endpoints also includes the recited endpoint values themselves (e.g. from 1.0 to 5.0 includes both 1.0 and 5.0). Any numerical range recited herein is intended to include all sub-ranges subsumed therein.

The particular features, structures, characteristics or embodiments may be combined in any suitable manner, as would be apparent to a person skilled in the art from this disclosure, in one or more embodiments.

The sensitivity is the percentage of patients suffering from a disease and who were tested as positive among a population of patients identified as suffering from said disease using a reference test. A test with 100% sensitivity will recognize all patients with the disease by testing them positive.

The specificity is the percentage of patients who do not suffer from a disease and who were tested as negative among a population of patients as not suffering from a disease using a reference test. A test with 100% specificity will exclude the disease from the patients not suffering from the disease.

Patients that are studied comprise patients suffering from Alzheimer's disease (AD), mental depression (PSY), dementia with Lewy bodies (DLB), frontotemporal dementia (FTD) and/or vascular dementia (VD). Among the group of patients suffering from Alzheimer's disease, two sub-groups are distinguished: those presenting a high concentration of noradrenaline (3000 pmol/1<[NA]<15000 pmol/l), referenced as AD1 patients, and those presenting a low concentration of noradrenaline (100 pmol/1<[NA]<3000 pmol/l), referenced as AD2 patients. As it will be understood at a later stage of the present description, the number of 3000 pmol/l is susceptible of variation and corresponds to the predetermined threshold which is used to differentiate between AD1 and AD2.

AD1 patients suffer from an early stage of Alzheimer's disease, while AD2 patients suffer from advanced stage of Alzheimer's disease.

Early AD (or AD1) corresponds to the stage in which the impairment of learning and memory has increased enough to establish a conventional definitive diagnosis. In this stage, the patient is usually capable of adequately communicating basic ideas. While performing fine motor tasks, such as writing, drawing or dressing herself/himself, certain movement coordination and planning difficulties may be present but they are commonly unnoticed.

Advanced AD (or AD2) corresponds to the last stage of the disease in which the patient is completely dependent upon caregivers. Language is reduced to simple phrases or even single words, eventually leading to complete loss of speech. Although aggressiveness can still be present, extreme apathy and exhaustion are much more common.

Another group of patients are patients which present cognitive problems, anxiety, burn-out, sleep apnea, and/or alcoholism has been selected. They are classified as neurological control (NC) and with the PSY patients, their medical condition is the least extreme among all the patients involved in this study.

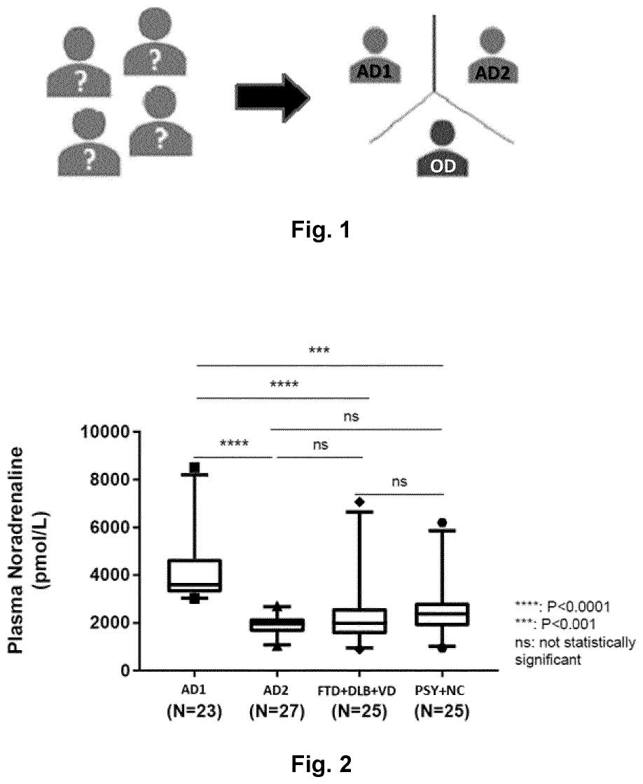

The concept of the differential diagnosis according to the invention is shown on FIG. 1 and are summarized below:
1. Clinical data, such as age and/or score to a medical questionnaire for screening cognitive function, are retrieved.
2. Level of catecholamines in the blood sample of the subject, preferentially in the plasma, are measured. The catecholamines which are analyzed are the adrenaline, the noradrenaline and the dopamine.
3. When the level of noradrenaline is lower or equal to a predetermined threshold, it is checked whether the subject suffers from arterial hypertension and to which extent.
4. The clinical data, the level of catecholamines and the arterial hypertension status (if determined) are combined together in a mathematical algorithm in order to calculate one global note in relation with each neurodegenerative disease. The global note also considers reference values.
5. Based on the global note obtained for each neurodegenerative disease, the clinician can determine whether the subject suffers from AD1, AD2, or OD (“OD” stands for “other dementia”, i.e., PSY, DLB, FTD, VD or mixed dementia).

The mathematical algorithm that has been established in the present case is actually an iterative algorithm. It refers to a mathematical procedure that generates a sequence of improving approximate solutions for a class of problems. Starting from a choice of an initial score considered as a first draft solution, the algorithm proceeds by iterations during which it determines a succession of refined approximate solution which get gradually closer to the searched solution.

Therefore, starting from 100 patients, and being aware of which disease said patients are suffering from, it has been possible to determine, with good accuracy, a mathematical algorithm which can now be used to diagnose a patient with an unknown disease selected among the diseases of the initial panel of patients. In other words, a retrospective exploratory clinical study has been carried out to provide the in vitro method for performing a differential diagnosis of neurodegenerative disease in one subject according to the present invention.

### Initial Panel of Patients Used to Determine the Mathematical Algorithm

The 100 patients have been clinically diagnosed by making use of the different clinical techniques in order to determined disease of the patients. The different clinical techniques to achieve such diagnosis can be briefly summarized by (1) proceeding to a clinical evaluation, such as investigating the response to one or more medical questionnaires, (2) performing and analyzing a lumbar puncture and (3) completing the evaluation by performing medical imaging studies, such as magnetic resonance imaging (MRI), DaTSCAN (process of neuro imaging the radiopharmaceutical drug ioflupane) and/or positron emission tomography (PET-SCAN).

Among the selected 100 patients, 50 patients have been clinically diagnosed as suffering from AD (23 suffering from AD1 and 27 suffering from AD2), 10 patients have been clinically diagnosed as suffering from FTD, 8 patients have been clinically diagnosed as suffering from DLB, 7 patients have been clinically diagnosed as suffering from VD, 6 patients have been clinically diagnosed as suffering from PSY and 19 patients have been clinically diagnosed as neurological control (NC).

### Criteria Used in the Method

The age of the subjects that are to be diagnosed is retrieved, either by clearly asking it to the subject himself/herself, and/or by asking it to one of his/her acquaintances, and/or by checking it in her/his identification papers.

The score to a medical questionnaire for screening cognitive function is determined. The medical questionnaire may preferentially be an abbreviated mental test (AMT) (see Hodkinson H. M., Evaluation of a mental test score for assessment of mental impairment in the elderly, Age and ageing, 1972, 1, 233), a mini-mental state examination (MMSE) (also named as Folstein test, see Folstein M. F., “Mini-mental state”, a practical method for grading the cognitive state of patients for the clinician, J. psychiat. Res., 1975, 12, 189-198) or a six-item cognitive impairment test (6CIT) (see Katzman R. et al., Validation of a short orientation-memory-concentration test of cognitive impairment, Am. J. Psychiatry, 1983, 140 (6), 734-739). Any other suitable medical questionnaires used to assess basic cognitive functions can be used. More preferentially, the medical questionnaire is a mini-mental state examination (MMSE). These tests consist in a set of several questions (generally 20 to 30 questions) which assess basic cognitive functions. It requires the individual taking the test to give basic personal information (i.e., name, address, etc.), to give an answer to simple question based on common knowledge (i.e. answering the question “who is the president of the USA?”), and to remember simple items of information such as a list of three words or names and addresses to recall later. According to the responses given by the subject, a score is given and allows the clinician to categorize the subject. For instance, AD1 patients, which suffer from an early stage of Alzheimer's disease, can achieve a good MMSE score, while AD2 patients, which suffer from an advanced stage of Alzheimer's disease have generally great difficulties to achieve a good MMSE score. This kind of questionnaire gives a good idea of the medical conditions of a subject but does not allow to be certain of the disease.

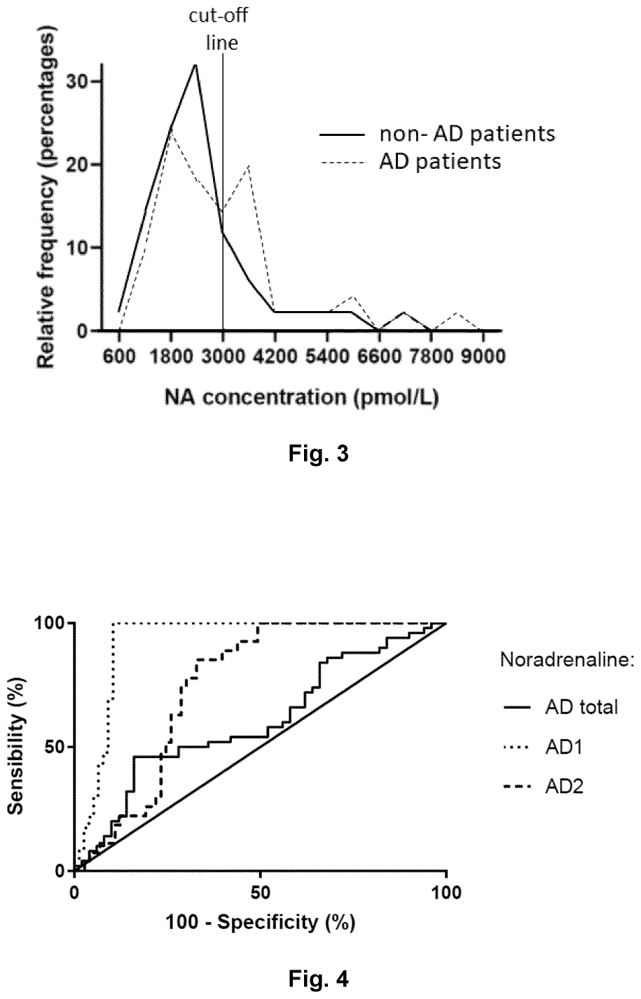

The sex of the subject can have an influence on the presence of a certain neurodegenerative disease. It has been indeed determined that women have a higher tendency to suffer from Alzheimer's disease that men (see Alzheimer's Association Report, Alzheimer's and Dementia, 2015, 11, 332-384, FIG. 3). The sex criterion could therefore be taken into account in order to adjust and/or refine the results of the in vitro method of the invention.

The presence of the APOEε4 allele in the genotype of the subject has been in the past an indication of the tendency of the patients to suffer from Alzheimer's disease (see Riedel B. C., J. Steroid Biochem. Mol. Biol., 2016, 160, 134-147). The number of copies of this APOEε4 allele in the genotype can be also taken into account.

In addition, the concentration of three catecholamines (CAs) in the blood sample of the patient is taken into account.

### Concentration of Catecholamines (CAs) in the Blood Sample

Techniques suitable to measure the level of a compound in a blood sample, preferentially in a plasma sample, can be used. Examples of suitable techniques include electrophoresis, in particular gel electrophoresis or capillary electrophoresis, spectrometry such as mass spectrometry or spectrofluorimetry, chromatography such as high-performance liquid chromatography (HPLC) or ultra-high-performance liquid chromatography (UHPLC) and their combinations (LC/MS, GC/MS), immunoassay techniques and electrochemistry-based techniques.

A sample of blood, preferentially of plasma, of the subject is analyzed in vitro and/or ex vivo. CAs were simultaneously quantified on plasma samples (1 mL/patient, conservation at −80° C.), using for example high-performance liquid chromatography coupled with electrochemical detection (HPLC-ECD) and Chromsystems kits (ref. 5000). HPLC-ECD is the reference method for CAs quantification in plasma (See F. A. J. Van der Hoorn et al., J. Chrom., 1989, 487, 17-28, in Determination of catecholamines in human plasma by high-performance liquid chromatography: comparison between a new method with fluorescence detection and an established method with electrochemical detection).

Data obtained by this method, namely the adrenaline concentration [A], the noradrenaline concentration [NA] and the dopamine concentration [D], are thus easy to obtain since the only biological fluid that is checked is the blood, which can be easily collected, contrary to the collection of samples in other biological fluids, such as cerebrospinal fluid (CSF).

It is also preferred that the subject is fasting at the moment of sampling, with preference at least 12 hours before sampling. This also ensures that that the subject is not affected by any medicines that can influence the arterial hypertension status.

It is preferred that the subject is maintained at rest during at least 30 minutes before the sampling, in order to allows her/his body fluid(s) to equilibrate. The equilibration time is considered important in order to regulate the level of the catecholamines in the blood.

Advantageously however, the subject can be in homeostatic imbalance when the sampling is performed. This means that it is not compulsory for the patients to respect said equilibration time by laying over at rest for at least 30 minutes before the sampling. The fact that it is not required to respect the equilibration time is a significant advantage, since it is not always easy to reserve an hospital bed for the patients and since it is also sometimes difficult to keep an elderly patient that is likely to suffer from a cognitive disease in a quiet mode during a certain amount of time. Moreover, it has been noted that when the equilibration time is respected, some of the patients can get scared, which is influential on the level of catecholamines in the blood. Sampling the subject when she/he is in homeostatic imbalance does not let the time to the subject to realize that she/he is going to be pricked.

### Low Concentration of Noradrenaline

A low concentration of noradrenaline is significative of a hypo-activity of the brain, specifically a low production of noradrenaline in the locus coeruleus of the brain. This nucleus in the brainstem is responsible for the brain synthesis of noradrenaline. This noradrenaline can in fact be released into the blood system through the adrenal gland. The hypo-activity of the brain, generally correlated with low score to the medical questionnaire for screening cognitive function, for instance with a low MMSE score, namely inferior to 15, may be an indication that the patient suffers from an advance stage of Alzheimer's disease (AD2).

Statistical studies, based notably on Receiver Operating Characteristic (ROC) curves, have allowed to determine a threshold with respect to the noradrenaline concentration in order to classify the subject as potentially suffering from AD1 or from AD2. ROC curves are graphical plot that illustrates the diagnosis ability of a binary classifier system as its discrimination threshold is varied. Indeed, the plasma noradrenaline data sample was not normally distributed and the histogram distribution of the samples did not show two groups of samples equally distributed (see FIG. 2). The relative frequency distribution of plasma noradrenaline concentration of AD patient versus non-AD patient is a visual way to determine a threshold with respect to the noradrenaline concentration in order to classify the subject as potentially suffering from AD1 or from AD2 (see FIG. 3). Indeed, it can be clearly seen that among all the patients with similar noradrenaline concentration, the AD patients are distributed into two distinct distribution, with a cut-off line comprised between 2900 pmol/l and 3100 pmol/l. The ROC analysis (see FIG. 4) has also allowed to identify the optimal cut-off that maximizes the Youden Index for the noradrenaline. The area under the curve (AUC) is not very good because the noradrenaline alone is not enough specific to distinguish between AD patients and non-AD patient. However, this cut-off, when applied to separate two sub-types of AD patients (AD1 and AD2), provides a very good discrimination (especially for AD1, since the AUC is the greatest among the one of AD1 and the one of AD2). The ROC curve analysis of plasma NA concentrations in AD versus non-AD patients has allowed to identify 3023 pmol/l as the optimal cut-off that maximises the Youden Index.

It is thus safe to affirm that when a subject has a noradrenaline concentration [NA] lower or equal to a predetermined threshold that is comprised between 2900 pmol/l and 3100 pmol/l, then the subject is suspected to suffer from an advanced stage of Alzheimer's disease (AD2). For practical feasibility, it is preferred that the cut-off for distinguishing between AD1 and AD2 is set to a noradrenaline concentration of at least 3000 pmol/l.

The suspicion is however not sufficient to base an accurate diagnosis on one patient. Further investigation is therefore needed to established an accurate and precise diagnosis.

### Arterial Hypertension

Once the concentration in noradrenaline has been determined in a subject, and in the case where this concentration is inferior or equal to the predetermined threshold, preferably inferior or equal to 3000 pmol/l, the arterial hypertension status (also referenced as the high blood pressure status, or HBP status) of the patient is determined.

To do so, it is checked whether the subject has blood pressure of at least 120/80 mmHg for at least three consecutive days. The first number corresponds to the systolic pressure and the second number corresponds to the diastolic pressure. Preferably, in order to be determined as suffering from arterial hypertension, the subject should present a blood pressure of at least 140/90 mmHg for at least three consecutive days. By comparison, the normal blood systolic pressure of a healthy patient is ranging between 90 and 119 mmHg while the normal blood diastolic pressure of a healthy patient is ranging between 60 and 79 mmHg.

In the case where the blood pressure is inferior to 140/90 mmHg during at least 3 consecutive days, preferentially inferior to 120/80 mmHg during at least 3 consecutive days, it can be concluded that the subject does not suffer from arterial hypertension.

### Determination of a Global Note for Each Neurodegenerative Disease

The experimental data (age, score, concentrations) are then processed with a mathematical algorithm in order to determine a global note which will be used to assess the final diagnosis. Experimental data such as sex and presence of the APOEε4 allele in the genotype can also be included. However, they can also be omitted. The determination of the global note first requires the determination of parameters.

As the subject to be diagnosed is, before the diagnosis, not categorized as suffering from one specific neurodegenerative disease in particular, but rather categorized as suffering from one of the medical conditions selected among six diseases (AD1, AD2, PSY, DLB, FTD, VD) in addition to the neurological control (NC), the mathematical algorithm has been derivatized in a way that each of the experimental clinical data (age, MMSE score, [A], [NA], [D] and extent of arterial hypertension in the case where [NA]>3000 pmol/l) is transformed into one parameter in function of one of the seven medical conditions that are studied in the present case.

That is the reason why for one subject presenting a [NA] superior to 3000 pmol/l, 35 parameters are determined. There is indeed no checking of the arterial hypertension of said subject.

However, when a subject has a [NA] inferior to 3000 pmol/l, the arterial hypertension is determined and therefore, there are a total of 42 parameters to be considered.

When the parameters are determined, they can be processed into a global note in relation with each of the neurological diseases under study. The global note thus computed will serve as basis to conclude on a final diagnosis for said subject.

### Determination of the Parameters

The clinician calculates parameters V_{disease}^{criterion} that correspond to the following mathematical equation (1):

$${V}_{d i s e a s e}^{criterion} = 1 + K \frac{V A {L}^{c r i t e r i o n} - {MINI}_{d i s e a s e}^{c r i t e r i o n}}{{MAXI}_{d i s e a s e}^{c r i t e r i o n} - {MINI}_{d i s e a s e}^{c r i t e r i o n}} \qquad ( 1 )$$

wherein VAL^{criterion} is the measured value of one criterion (i.e. age, score to the medical questionnaire, [A], [NA] and [D]).
wherein MINI_{disease}^{criterion} is the minimum reference value with respect to each neurodegenerative disease in function of each criterion (see for instance table I)
wherein MAXI_{disease}^{criterion} is the maximum reference value with respect to each neurodegenerative disease in function of each criterion (see for instance table II), and
wherein K is a mathematical parameter, K being comprised between 0.01 and 1.00, preferentially between 0.02 and 0.99, more preferentially between 0.05 and 0.95.

In a preferred embodiment; the reference values used in step (c) in relation with early stage of Alzheimer's disease (AD1) and implemented in equation (1) are as followed:
with respect to the age of said subject, the minimum reference value is at least 50 years and/or at most 60 years, preferably at least 51 year, more preferably at least 52 year and most preferably is 52.92; and/or the maximum reference value is at least 85 years and/or at most 90 years, preferably at most 89 years, more preferably at most 88 years, and most preferably is 87.72 years;
with respect to the score of said subject to a questionnaire adapted for screening cognitive function, the minimum reference value is at least 14 and/or at most 16, preferably is 15; and/or the maximum reference value is at least 27 and/or at most 29, preferably is 28;
with respect to the dopamine concentration [D], the minimum reference value is at least 90 pmol/l and/or at most 150 pmol/l, preferably at least 100 pmol/l, more preferably at least 105 pmol/l, and most preferably is 110.74 pmol/l; and/or the maximum reference value is at least 800 pmol/l and/or at most 1000 pmol/l, preferably at most 950 pmol/l, more preferably at most 925 pmol/l and most preferably is 878.22 pmol/l;
with respect to the adrenaline concentration [A] the minimum reference value is at least 40 pmol/l and/or at most 100 pmol/l, preferably at least 50 pmol/l, more preferably at least 60 pmol/l, and most preferably is 65.66 pmol/l; and/or the maximum reference value is at least 1500 pmol/l and/or at most 2000 pmol/l, preferably at most 1900 pmol/l, more preferably at most 1800 pmol/l and most preferably is 1720.74 pmol/l;
with respect to the noradrenaline concentration [NA] the minimum reference value is at least 2900 pmol/l and/or at most 3200 pmol/l, preferably at least 2950 pmol/l, more preferably at least 2960 pmol/l, and most preferably is 2962.54 pmol/l; and/or the maximum reference value is at least 8100 pmol/l and/or at most 9000 pmol/l, preferably at most 8800 pmol/l, more preferably at most 8700 pmol/l and most preferably is 8681.22 pmol/l.

In a preferred embodiment, the reference values used in step (c) in relation with advanced stage of Alzheimer's disease (AD2) and implemented in equation (1) are as followed:
with respect to the age of said subject, the minimum reference value is at least 45 years and/or at most 55 years, preferably at least 47 year, more preferably at least 50 year and most preferably is 51.94; and/or the maximum reference value is at least 85 years and/or at most 95 years, preferably at most 92 years, more preferably at most 90 years, and most preferably is 89.76 years;
with respect to the score of said subject to a questionnaire adapted for screening cognitive function, the minimum reference value is at least 3 and/or at most 5, preferably is 4; and/or the maximum reference value is at least 28 and/or at most 30, preferably is 29;
with respect to the dopamine concentration [D], the minimum reference value is at least 140 pmol/l and/or at most 175 pmol/l, preferably at least 145 pmol/l, more preferably at least 150 pmol/l, and most preferably is 152.88 pmol/l; and/or the maximum reference value is at least 500 pmol/l and/or at most 1000 pmol/l, preferably at most 800 pmol/l, more preferably at most 700 pmol/l and most preferably is 655.86 pmol/l;
with respect to the adrenaline concentration [A] the minimum reference value is at least 80 pmol/l and/or at most 200 pmol/l, preferably at least 120 pmol/l, more preferably at least 130 pmol/l, and most preferably is 143.08 pmol/l; and/or the maximum reference value is at least 1100 pmol/l and/or most 1500 pmol/l, preferably at most 1450 pmol/l, more preferably at most 1400 pmol/l and most preferably is 1371.09 pmol/l;
with respect to the noradrenaline concentration [NA] the minimum reference value is at least 750 pmol/l and/or at most 1150 pmol/l, preferably at least 850 pmol/l, more preferably at least 1000 pmol/l, and most preferably is 1023.12 pmol/l; and/or the maximum reference value is at least 2600 pmol/l and/or at most 3000 pmol/l, preferably at most 2900 pmol/l, more preferably at most 2800 pmol/l and most preferably is 2758.08 pmol/l.

In a preferred embodiment, the reference values used in step (c) in relation with mental depression (PSY) and implemented in equation (1) are as followed:
with respect to the age of said subject, the minimum reference value is at least 35 years and/or at most 45 years, preferably at least 37 year, more preferably at least 40 year and most preferably is 40.18; and/or the maximum reference value is at least 70 and/or at most 80 years, preferably at most 78 years, more preferably at most 76 years, and most preferably is 74.46 years;
with respect to the score of said subject to a questionnaire adapted for screening cognitive function, the minimum reference value is at least 21 and/or at most 23, preferably is 22; and/or the maximum reference value is at least 28 and/or at most 30, preferably is 29;
with respect to the dopamine concentration [D], the minimum reference value is at least 250 pmol/l and/or at most 320 pmol/l; preferably at least 260 pmol/l, more preferably at least 270 pmol/l, and most preferably is 276.36 pmol/l; and/or the maximum reference value is at least 450 pmol/l and/or at most 750 pmol/l, preferably at most 700 pmol/l, more preferably at most 600 pmol/l and most preferably is 519.18 pmol/l;
with respect to the adrenaline concentration [A] the minimum reference value is at least 180 pmol/l and/or at most 220 pmol/l; preferably at least 185 pmol/l, more preferably at least 190 pmol/l, and most preferably is 191.10 pmol/l; and/or the maximum reference value is at least 360 pmol/l and/or at most 450 pmol/l, preferably at most 420 pmol/l, more preferably at most 410 pmol/l and most preferably is 406.98 pmol/l;
with respect to the noradrenaline concentration [NA] the minimum reference value is at least 1000 pmol/l and/or at most 2000 pmol/l; preferably at least 1200 pmol/l, more preferably at least 1400 pmol/l, and most preferably is 1533.70 pmol/l; and/or the maximum reference value is at least 3000 pmol/l and/or at most 4000 pmol/l, preferably at most 3800 pmol/l, more preferably at most 3700 pmol/l and most preferably is 3691.38 pmol/l.

In a preferred embodiment, the reference values used in step (c) in relation with dementia with Lewy bodies (DLB) and implemented in equation (1) are as followed:
with respect to the age of said subject, the minimum reference value is at least 55 years and/or at most 65, preferably at least 57 year, more preferably at least 60 year and most preferably is 61.74; and/or the maximum reference value is at least 75 years and/or at most 85 years, preferably at most 83 years, more preferably at most 82 years, and most preferably is 81.6 years;
with respect to the score of said subject to a questionnaire adapted for screening cognitive function, the minimum reference value is at least 7 and/or at most 9, preferably is 8; and/or the maximum reference value is at least 23 and/or most 25, preferably is 24;
with respect to the dopamine concentration [D], the minimum reference value is at least 150 pmol/l and/or at most 250 pmol/l; preferably at least 170 pmol/l, more preferably at least 180 pmol/l, and most preferably is 203.84 pmol/l; and/or the maximum reference value is at least 300 pmol/l and/or at most 400 pmol/l, preferably at most 390 pmol/l, more preferably at most 380 pmol/l and most preferably is 375.36 pmol/l;
with respect to the adrenaline concentration [A] the minimum reference value is at least 80 pmol/l and/or at most 125 pmol/l; preferably at least 90 pmol/l, more preferably at least 100 pmol/l, and most preferably is 108 pmol/l; and/or the maximum reference value is at least 580 pmol/l and/or at most 720 pmol/l, preferably at most 690 pmol/l, more preferably at most 670 pmol/l and most preferably is 650 pmol/l;
with respect to the noradrenaline concentration [NA] the minimum reference value is at least 900 pmol/l and/or at most 1300 pmol/l; preferably at least 1000 pmol/l, more preferably at least 1050 pmol/l, and most preferably is 1065.26 pmol/l; and/or the maximum reference value is at least 6500 pmol/l and/or at most 8000 pmol/l, preferably at most 7500 pmol/l, more preferably at most 7300 pmol/l and most preferably is 7216.50 pmol/l.

In a preferred embodiment, the reference values used in step (c) in relation with frontotemporal dementia (FTD) and implemented in equation (1) are as followed:
with respect to the age of said subject, the minimum reference value is at least 45 years and/or at most 53, preferably at least 47 year, more preferably at least 48 year and most preferably is 49; and/or the maximum reference value is at least 70 years and/or at most 80 years, preferably at most 78 years, more preferably at most 76 years, and most preferably is 75.48 years;
with respect to the score of said subject to a questionnaire adapted for screening cognitive function, the minimum reference value is at least 0 and/or at most 1, preferably is 0; and/or the maximum reference value is at least 26 and/or most 28, preferably is 27;
with respect to the dopamine concentration [D], the minimum reference value is at least 200 pmol/l and/or at most 400 pmol/l; preferably at least 230 pmol/l, more preferably at least 240 pmol/l, and most preferably is 245.98 pmol/l; and/or the maximum reference value is at least 900 pmol/l and/or at most 1100 pmol/l, preferably at most 1050 pmol/l, more preferably at most 1020 pmol/l, and most preferably is 1002.66 pmol/l;
with respect to the adrenaline concentration [A] the minimum reference value is at least 100 pmol/l and/or at most 150 pmol/l; preferably at least 110 pmol/l, more preferably at least 120 pmol/l, and most preferably is 123.48 pmol/l; and/or the maximum reference value is at least 1100 pmol/l and/or at most 1500 pmol/l, preferably at most 1400 pmol/l, more preferably at most 1300 pmol/l and most preferably is 1270.90 pmol/l;
with respect to the noradrenaline concentration [NA] the minimum reference value is at least 700 pmol/l and/or at most 1000 pmol/l; preferably at least 800 pmol/l, more preferably at least 850 pmol/l, and most preferably is 874.16 pmol/l; and/or the maximum reference value is at least 5300 pmol/l and/or at most 6000 pmol/l, preferably at most 5900 pmol/l, more preferably at most 5800 pmol/l and most preferably is 5743.62 pmol/l.

In a preferred embodiment, the reference values used in step (c) in relation with vascular dementia (VD) and implemented in equation (1) are as followed:
with respect to the age of said subject, the minimum reference value is at least 55 years and/or at most 63, preferably at least 56 year, more preferably at least 58 year and most preferably is 58.8; and/or the maximum reference value is at least 80 years and/or at most 90 years, preferably at most 88 years, more preferably at most 86 years, and most preferably is 85.68 years;
with respect to the score of said subject to a questionnaire adapted for screening cognitive function, the minimum reference value is at least 20 and/or at most 22, preferably is 21; and/or the maximum reference value is at least 27 and/or most 29, preferably is 28;
with respect to the dopamine concentration [D], the minimum reference value is at least 140 pmol/l and/or at most 250 pmol/l; preferably at least 160 pmol/l, more preferably at least 180 pmol/l, and most preferably is 198.94 pmol/l; and/or the maximum reference value is at least 450 pmol/l and/or at most 580 pmol/l, preferably at most 560 pmol/l, more preferably at most 540 pmol/l, and most preferably is 533.46 pmol/l;
with respect to the adrenaline concentration [A] the minimum reference value is at least 200 pmol/l and/or at most 270 pmol/l; preferably at least 210 pmol/l, more preferably at least 230 pmol/l, and most preferably is 240.10 pmol/l; and/or the maximum reference value is at least 1200 pmol/l and/or at most 1800 pmol/l, preferably at most 1700 pmol/l, more preferably at most 1600 pmol/l and most preferably is 1525.92 pmol/l;
with respect to the noradrenaline concentration [NA] the minimum reference value is at least 1400 pmol/l and/or at most 1800 pmol/l; preferably at least 1450 pmol/l, more preferably at least 1500 pmol/l, and most preferably is 1518.02 pmol/l; and/or the maximum reference value is at least 3400 pmol/l and/or at most 4000 pmol/l, preferably at most 3900 pmol/l, more preferably at most 3800 pmol/l and most preferably is 3705.66 pmol/l.

In a preferred embodiment, the step (c) further comprises calculating one global note in relation with neurological control (NC); with preference, the reference values used in step (c) in relation with neurological control (NC) and implemented in equation (1) are as followed:
with respect to the age of said subject, the minimum reference value is at least 35 years and/or at most 45, preferably at least 37 year, more preferably at least 39 year and most preferably is 40.18; and/or the maximum reference value is at least 80 years and/or at most 90 years, preferably at most 88 years, more preferably at most 86 years, and most preferably is 84.66 years;
with respect to the score of said subject to a questionnaire adapted for screening cognitive function, the minimum reference value is at least 22 and/or at most 24, preferably is 23; and/or the maximum reference value is at least 29 and/or most 30, preferably is 30;
with respect to the dopamine concentration [D], the minimum reference value is at least 180 pmol/l and/or at most 240 pmol/l; preferably at least 190 pmol/l, more preferably at least 200 pmol/l, and most preferably is 207.76 pmol/l; and/or the maximum reference value is at least 1250 pmol/l and/or at most 1600 pmol/l, preferably at most 1500 pmol/l, more preferably at most 1400 pmol/l, and most preferably is 1334.16 pmol/l;
with respect to the adrenaline concentration [A] the minimum reference value is at least 110 pmol/l and/or at most 200 pmol/l; preferably at least 130 pmol/l, more preferably at least 150 pmol/l, and most preferably is 163.66 pmol/l; and/or the maximum reference value is at least 2250 pmol/l and/or at most 2500 pmol/l, preferably at most 2450 pmol/l, more preferably at most 2400 pmol/l and most preferably is 2366.40 pmol/l;
with respect to the noradrenaline concentration [NA] the minimum reference value is at least 900 pmol/l and/or at most 1650 pmol/l; preferably at least 910 pmol/l, more preferably at least 920 pmol/l, and most preferably is 925.12 pmol/l; and/or the maximum reference value is at least 6000 pmol/l and/or at most 7000 pmol/l, preferably at most 6600 pmol/l, more preferably at most 6400 pmol/l and most preferably is 6331.14 pmol/l.

Tables I and II respectively show the preferred combination of minimum reference values and the preferred combination of maximum reference values that have been determined with respect to the five criteria (i.e. age, score to the medical questionnaire, [A], [NA] and [D]) and in relation with each neurodegenerative disease that were taken into consideration in this study. These reference values have been determined on the initial panel of 100 patients with known diseases.

TABLE I
Reference table showing the lowest value of each criterion in
function of the neurodegenerative disease. The age is given in years
and the concentrations are given in pmol/l. The numbers under the
MMSE score correspond to the marks obtained at this questionnaire.

| MINI_{disease}^{criterion} | Age | MMSE score | [NA] | [A] | [D] |
|---|---|---|---|---|---|
| AD1 | 52.92 | 15 | 2962.54 | 65.66 | 110.74 |
| AD2 | 51.94 | 4 | 1023.12 | 143.08 | 152.88 |
| PSY | 40.18 | 22 | 1533.7 | 191.1 | 276.36 |
| DLB | 61.74 | 8 | 1065.26 | 103.88 | 203.84 |
| FTD | 49.00 | 0 | 874.16 | 123.48 | 245.98 |
| VD | 58.80 | 21 | 1518.02 | 240.1 | 198.94 |
| NC | 40.18 | 23 | 925.12 | 163.66 | 207.76 |

TABLE II
Reference table showing the highest value of each criterion in
function of the neurodegenerative disease. The age is given in years and
the concentrations are given in pmol/l. The numbers under the MMSE
score correspond to the marks obtained at this questionnaire.

| MAXI_{disease}^{criterion} | Age | MMSE score | [NA] | [A] | [D] |
|---|---|---|---|---|---|
| AD1 | 87.72 | 28 | 8621.22 | 1720.74 | 878.22 |
| AD2 | 89.76 | 29 | 2758.08 | 1371.9 | 655.86 |
| PSY | 74.46 | 29 | 3691.38 | 406.98 | 519.18 |
| DLB | 81.60 | 24 | 7216.5 | 418.2 | 375.36 |
| FTD | 75.48 | 27 | 5743.62 | 1270.92 | 1002.66 |
| VD | 85.68 | 28 | 3705.66 | 1525.92 | 533.46 |
| NC | 84.66 | 30 | 6331.14 | 2366.4 | 1334.16 |

It is to be noted the following:

if VAL^{criterion}<MINI_{disease}^{criterion} or if VAL^{criterion}>MAXI_{disease}^{criterion} then V_{disease}^{criterion}=0.

Stated in other words, this means that if the subject to be diagnosed, with regard to one specific criterion, presents a measured value which is outside the range of the references values, then the parameter is set to zero.

On the other hand, if the measured value is inside the range of the reference values, the mathematic equation (1) is used.

Advantageously, a correction factors can be attributed to these lowest and highest reference values. Said correction factors can be preferentially comprised between 0.50 and 1.50. More preferentially, said correction factors can be comprised between 0.75 and 1.25. If the correction factors is equal to 1, then the reference values are not corrected.

The correction factors is advantageously used to take into consideration any possible deviation(s) than can occur during the determination of the levels of CAs and/or the possible errors made by inadvertence at the determination of the score to the medical questionnaire. Iteration of the diagnosis method of the invention on one subject with different reference tables obtained thanks to different correction factors s can thus provide better statistical robustness of the results. For instance, if a subject is diagnosed with dementia with Lewy bodies with three different reference tables, then the certainty of the diagnosis is increased in comparison with the case where said subject would have been diagnosed as suffering from this same specific disease by reference to only one table.

With regard to the arterial hypertension status, a determination is carried out when the noradrenaline blood concentration [NA] of said subject is inferior or equal to a predetermined threshold, preferentially inferior or equal to 3000 pmol/l. The arterial hypertension status can be dependent on the age of the said subject, which has for consequence that the parameter V^{HT} is ranging between 0.1 and 5.0. In fact, the parameter V^{HT} can be determined according the following mathematical equation (2)

V^{HT}=α(age)+β (2)

wherein α is a variable in function of the age of the subject, having values comprised between 0.0 and 10.0. When the subject is less than 70 years old, α equals 0. When the subject is more than 85 years old, α is superior or equal to 5. When the subject is more than 95 years old, α is superior or equal to 8;
and, wherein β is a variable in function of the seriousness of the arterial hypertension status, having values comprised between 0 and 1.0, 0 being significative of a subject not presenting symptoms of arterial hypertension and 1.0 being significative of a subject presenting severe signs of said medical conditions.

### Determination of the Global Note

As the clinician has now in hand, for one subject to be diagnosed, one parameter per criterion per neurodegenerative disease, the clinician will compute a global note that will help to attribute a decision with regard to the medical conditions of the subject. The global note is thus established in function of each neurodegenerative disease or medical conditions that are actually studied. In the present case, seven global notes are thus computed.

With respect to each neurodegenerative disease, seven global notes (one global note per medical conditions) are calculated according to mathematical equation (3):

GLOBAL NOTE_{disease}=w₁V_{age}+w₂V_{score}+w₁(V_{[A]}+V_{[NA]}+V_{[D]}) (3)

wherein w₁ and w₂ are weighting factor,
wherein V_{age} is the parameter obtained with respect to the age of said subject in function with the neurodegenerative disease under consideration,
wherein V_{score} is the parameter obtained with respect to the score of said subject to the medical questionnaire in function of the neurodegenerative disease under consideration,
wherein V_{[A]} is the parameter obtained with respect to the adrenaline blood concentration [A] of said subject in function of the neurodegenerative disease under consideration,
wherein V_{[NA]} is the parameter obtained with respect to the noradrenaline blood concentration [NA] of said subject in function of the neurodegenerative disease under consideration, and
wherein V_{[D]} is the parameter obtained with respect to the dopamine blood concentration [D] of said subject in function of the neurodegenerative disease under consideration.

When the subject is suspected of suffering from AD2, namely when the noradrenaline concentration [NA] has been determined as being inferior or equal to a predetermined threshold, preferentially inferior or equal to 3000 pmol/l, then, the mathematical formula of the global note for this specific disease is established according to equation (4):

GLOBAL NOTE_{AD2}=w₁V_{age}+w₂V_{score}+w₁(V_{[A]}+V_{[NA]}+V_{[D]})+V^{HT} (4)

wherein V^{HT} is the parameter obtained with respect to the arterial hypertension of said subject in function of the neurodegenerative disease under consideration and determined with equation (2).

In the calculation of the global note, the clinician takes into consideration that the parameters with respect to the score are less relevant than the parameters in relation with the age or the concentrations. The determination of the age and the biochemical determination of concentrations are indeed scientifically less subjective than the determination of a score (which at the end consists in asking question to one individual). This degree of variability is implemented in the algorithm by establishing a first weighting factor w1 with respect to parameter linked to the age and to the catecholamine concentration different to a second weighting factor w2 which is attributed to the parameters linked to the score of the medical questionnaire.

w₁ is preferentially a number comprised between 1.0 and 10.0, more preferentially a number comprised between 2.0 and 9.0. In one instance, w₁ is equal to 5.

w₂ is preferentially a number comprised between 0.1 and 3.0, more preferentially a number comprised between 0.5 and 2.5. In one instance, w₂ is equal to 2.

Seven global notes (one global note for each medical conditions) are therefore determined for each subject. In this case, six parameters age, MMSE score, [A], [NA], [D], and arterial hypertension status are considered in order to determine the differential diagnosis.

### Decision Supported by the Differential Diagnosis

The clinician will then compare the seven global notes. For one subject, the conclusion will be that she/he is suffering from the disease having the highest global notes. The clinician could attribute a second-best diagnosis or even a third-best diagnosis, always based on the global notes. That is how a subject suffering from mixed dementia can be diagnosed.

### Computer for Carrying Out the In Vitro Method for Performing a Differential Diagnosis of Neurodegenerative Disease

Once step (a) of the method has been carried out, the data can advantageously be implemented into a computer. The step (b) of comparing the noradrenaline concentration to a predetermined threshold is then carried out by a computer. The computer then calculates one global note per each neurodegenerative disease according to step (c) and then determines, based on each global note per neurodegenerative disease, whether said subject suffers from one or more neurodegenerative diseases selected from early stage Alzheimer's disease (AD1), advanced stage of Alzheimer's disease (AD2), mental depression (PSY), dementia with Lewy bodies (DLB), frontotemporal dementia (FTD), and/or vascular dementia (VD).

### Example

The present invention will be better understood by way of the example below.

Table III presents the experimental clinical data that have been retrieved from 15 subjects.

As explained above, when the noradrenaline concentration has been determined to be low, namely inferior or equal to the threshold of 3000 pmol/l, arterial hypertension of the subject, as additional criterion, is checked. The results given in table III show dichotomously (YES or NO) the status of the arterial hypertension to the subject.

TABLE III
Clinical data determined for 15 subjects according to the
method of the invention. The age is expressed in years,
the MMSE score is expressed in marks,
the catecholamines concentrations are expressed
in pmol/l. When [NA] is lower than 3000
pmol/l, the arterial hypertension of the subject
has been determined. The arterial
hypertension has not been checked for subjects 1, 2
and 12, since [NA] is superior to 3000 pmol/l.

| | Medical | | MMSE | | | | Arterial |
|---|---|---|---|---|---|---|---|
| | condition | Age | score | [NA] | [A] | [D] | hypertension |
| 1 | AD1 | 83 | 25 | 3208 | 67 | 341 | / |
| 2 | AD1 | 77 | 23 | 3600 | 961 | 861 | / |
| 3 | AD2 | 73 | 24 | 2054 | 1345 | 302 | NO |
| 4 | AD2 | 75 | 22 | 2060 | 378 | 403 | NO |
| 5 | PSY | 41 | 26 | 2324 | 195 | 382 | NO |
| 6 | PSY | 52 | 22 | 2368 | 257 | 317 | NO |
| 7 | DLB | 68 | 21 | 1174 | 253 | 287 | YES |
| 8 | DLB | 73 | 21 | 2497 | 410 | 223 | YES |
| 9 | FTD | 59 | 18 | 2408 | 389 | 431 | NO |
| 10 | FTD | 69 | 24 | 1248 | 684 | 983 | YES |
| 11 | FTD | 63 | 27 | 1614 | 759 | 410 | NO |
| 12 | FTD | 74 | 14 | 5631 | 353 | 314 | / |
| 13 | VD | 79 | 25 | 2470 | 291 | 336 | YES |
| 14 | NC | 41 | 23 | 2229 | 263 | 212 | NO |
| 15 | NC | 49 | 24 | 1276 | 388 | 354 | NO |

As explained above, the patients having noradrenaline concentration below the threshold of 3000 pmol/l are suspected of suffering from an advance stage of the Alzheimer's disease (AD2).

With these experimental clinical data in hand, the clinician will process the mathematical algorithm that has been described above.

A parameter is thus determined in accordance with the five or six criteria and the seven medical conditions that are searched in this study. Accordingly, subjects 1, 2 and 12 present only five criteria, since the arterial hypertension has not been checked.

In one detailed example, for instance in subject number 3, the global notes are calculated as followed:

The input data are summarized:

age: 73 years; MMSE: 24; [NA]=2054 pmol/l; [A]=1345 pmol/l; [D]=302 (see table III)
The lowest and the highest reference values are respectively listed in tables I and II. In this example, the reference values have not been corrected (thus 6=1).

Using equation (1) in which the value of K is 0.05, the parameters and the global notes are calculated as followed:

With Respect to AD1

V^{age}=1+K[(73−52.92)/(87.72−52.92)]=1.029

V^{MMSE}=1+K[(24−15)/(28−15)]=1.034

V^{[NA]}=0 because 2054 is <2962.54

V^{[A]}=1+K[(1345−65.66)/(1720.74−65.66)]=1.039

V^{[D]}=1+K[(302−110.74)/(878.22−110.74)]=1.012

Using the value of w₁=5 and w₂=2, the global note with respect to AD1 has been determined thanks to equation (3), as followed:

GLOBAL NOTE_{AD1}=w₁1.029+w₂1.034+w₁0+w₁1.039+w₁1.012=17.468 rounded to 17.47.

With Respect to AD2

V^{age}=1+K[(73−51.94)/(89.76−51.94)]=1.028

V^{MMSE}=1+K[(24−4)/(29−4)]=1.039

V^{[NA]}=1+K[(2054−1023.12)/(2758.08−1023.12)]=1.030

V^{[A]}=1+K[(1345−143.08)/(1371.9−143.08)]=1.049

V^{[D]}=1+K[(302−152.88)/(655.86−152.88)]=1.015

Using the value of w₁=5 and w₂=2, the global note with respect to AD2 has been determined thanks to equation (3), as followed:

GLOBAL NOTE_{AD2}=w₁1.028+w₂1.039+w₁1.030+w₁1.049+w₁1.015=22.688 rounded to 22.69.

Considering that 2054 pmol/l is inferior to the predetermined threshold (which can be 3000 pmol/l), a parameter related to the hypertension status of the subject has been determined according to equation (2). The subject does not present any symptom of hypertension (then β=0). As the subject is older than 70 years old but younger than 85 years old, the parameter α=3.

Therefore, according to equation (4), the global note considering the arterial hypertension status with respect to AD2 is:

GLOBAL NOTE_{AD2}=22.69+3=25.69.

With Respect to PSY

V^{age}=1+K[(73−40.18)/(74.46−40.18)]=1.048

V^{MMSE}=1+K[(24−22)/(29−22)]=1.014

V^{[NA]}=1+K[(2054−1533.7)/(3691.38−1533.7)]=1.012

V^{[A]}=0 because 1345 is >406.98

V^{[D]}=1+K[(302−276.36)/(519.18−276.36)]=1.005

Using the value of w₁=5 and w₂=2, the global note with respect to PSY has been determined thanks to equation (3), as followed:

GLOBAL NOTE_{PSY}=w₁1.048+w₂1.014+w₁1.012+w₁0+w₁1.005=17.353 rounded to 17.35.

With Respect to DLB

V^{age}=1+K[(73−61.74)/(81.6−61.74)]=1.028

V^{MMSE}=1+K[(24−8)/(24−8)]=1.050

V^{[NA]}=1+K[(2054−1065.26)/(7216.5−1065.26)]=1.008

V^{[A]}=0 because 1345 is >418.2

V^{[D]}=1+K[(302−203.84)/(375.36−203.84)]=1.029

Using the value of w₁=5 and w₂=2, the global note with respect to DLB has been determined thanks to equation (3), as followed:

GLOBAL NOTE_{DLB}=w₁1.028+w₂1.050+w₁1.008+w₁0+w₁1.029=17.425 rounded to 17.42.

With Respect to FTD

V^{age}=1+K[(73−49)/(75.48−49)]=1.045

V^{MMSE}=1+K[(24−0)/(27−0)]=1.044

V^{[NA]}=1+K[(2054−874.16)/(5743.62−874.16)]=1.012

V^{[A]}=0 because 1345 is >1270.92

V^{[D]}=1+K[(302−245.98)/(1002.66−245.98)]=1.003

Using the value of w₁=5 and w₂=2, the global note with respect to FTD has been determined thanks to equation (3), as followed:

GLOBAL NOTE_{FTD}=w₁1.045+w₂1.044+w₁1.012+w₁0+w₁1.003=17.388 rounded to 17.39.

With Respect to VD

V^{age}=1+K[(73−58.8)/(85.68−58.8)]=1.026

V^{MMSE}=1+K[(24−21)/(28−21)]=1.021

V^{[NA]}=1+K[(2054−1518.02)/(3705.66−1518.02)]=1.012

V^{[A]}=1+K[(1345−240.1)/(1525.92−240.1)]=1.043

V^{[D]}=1+K[(302−198.94)/(533.46−198.94)]=1.015

Using the value of w₁=5 and w₂=2, the global note with respect to VD has been determined thanks to equation (3), as followed:

GLOBAL NOTE_{VD}=w₁1.026+w₂1.021+w₁1.012+w₁1.043+w₁1.015=22.522 rounded to 22.52.

With Respect to NC

V^{age}=1+K[(73−40.18)/(84.66−40.18)]=1.037

V^{MMSE}=1+K[(24−23)/(30−24)]=1.008

V^{[NA]}=1+K[(2054−925.12)/(6331.14−925.12)]=1.010

V^{[A]}=1+K[(1345−163.66)/(2366.4−163.66)]=1.027

V^{[D]}=1+K[(302−207.76)/(1334.16−207.76)]=1.004

Using the value of w₁=5 and w₂=2, the global note with respect to NC has been determined thanks to equation (3), as followed:

GLOBAL NOTE_{NC}=w₁1.037+w₂1.008+w₁1.010+w₁1.027+w₁1.004=22.406 rounded to 22.41.

In conclusion, for the subject number 3, the highest global note is GLOBAL NOTE_{AD2} (22.69) without taking into account the refinement of using the hypertension status. This result has in fact confirmed the medical condition of this subject that has been determined using conventional methods. Since the global note obtained when considering the arterial hypertension status is higher (25.69), there are no changes to the diagnosis, since the global note in relation with AD2 stays the highest among all the calculated global notes.

The global notes in relation with each neurodegenerative disease obtained for the 15 subjects under consideration are indicated in Table IV. The same calculation as above has been made for obtaining those global notes.

TABLE IV
Global notes in relation with each medical condition and
associated diagnosis compared to the clinical conditions for 15 patients. Column Xla of the diagnosis
refers to a diagnosis established without considering arterial hypertension
while column Xlb of the diagnosis refers to a diagnosis established by refinement with
consideration of arterial hypertension.

| | | | | | | | | | X | XI | |
|---|---|---|---|---|---|---|---|---|---|---|---|
| I | II | III | IV | V | VI | VII | VIII | IX | Clinical | Diagnosis | |
| | AD1 | AD2 | AD2* | PSY | DLB | FTD | VD | NC | | Xla | Xlb |
| 1 | 22.38 | 12.38 | / | 12.30 | 10.29 | 12.24 | 17.58 | 17.40 | AD1 | AD1 | / |
| 2 | 22.64 | 12.41 | / | 7.25 | 12.39 | 17.61 | 17.58 | 22.57 | AD1 | AD1 | / |
| 3 | 17.47 | 22.69 | 25.69 | 17.35 | 17.42 | 17.39 | 22.52 | 22.41 | AD2 | AD2 | AD2 |
| 4 | 17.35 | 22.55 | 25.55 | 17.41 | 17.51 | 22.49 | 22.41 | 20.31 | AD2 | AD2 | AD2 |
| 5 | 12.16 | 17.35 | 17.35 | 22.16 | 15.24 | 17.20 | 12.22 | 22.13 | PSY | PSY | PSY |
| 6 | 12.15 | 22.37 | 22.37 | 22.30 | 17.43 | 22.24 | 17.20 | 20.17 | PSY | AD2 | AD2 |
| 7 | 17.24 | 22.18 | 22.28 | 15.28 | 22.40 | 22.31 | 17.15 | 20.19 | DLB | DLB | DLB |
| 8 | 17.28 | 22.41 | 22.51 | 10.35 | 22.55 | 17.45 | 22.29 | 20.29 | DLB | DLB | DLB |
| 9 | 17.21 | 22.49 | 22.49 | 20.63 | 12.34 | 22.36 | 20.31 | 20.25 | FTD | AD2 | AD2 |
| 10 | 12.28 | 17.23 | 17.33 | 7.24 | 12.20 | 22.66 | 12.22 | 22.42 | FTD | FTD | FTD |
| 11 | 17.37 | 22.50 | 22.50 | 17.38 | 10.04 | 22.46 | 22.39 | 22.33 | FTD | AD2 | AD2 |
| 12 | 20.38 | 17.31 | / | 15.47 | 22.74 | 22.60 | 15.25 | 20.45 | FTD | DLB | / |
| 13 | 17.37 | 19.49 | 22.59 | 17.33 | 20.61 | 17.24 | 22.47 | 22.36 | VD | VD | AD2 |
| 14 | 12.12 | 17.30 | 17.30 | 17.18 | 17.28 | 12.18 | 17.12 | 22.08 | NC | NC | NC |
| 15 | 12.20 | 17.27 | 17.27 | 17.40 | 17.55 | 22.20 | 12.19 | 22.14 | NC | FTD | FTD |

For all the subjects except subjects 1, 2 and 12, the global note in relation with AD2 has been calculated twice, once using equation (3) and shown in column III and once using equation (4) and shown in column IV, and thus taking into account the parameter linked to the arterial hypertension.

For subjects 1 and 2, which were presenting a high level of noradrenaline concentration (>3000 pmol/l), the mathematical processing of the clinical data has confirmed that they suffer from AD1, namely an early stage of Alzheimer's disease. Indeed, the global note calculated using equation (3) has given the highest global notes for AD1 (22.38 and 22.64, respectively).

For subject 12, the mathematical processing of the clinical data has provided another diagnosis than the one clinically established. The subject is actually suffering from DLB instead of FTD, since the global note in relation with DLB (22.74) is higher than the global note in relation with FTD (22.60). In fact, FTD becomes the second-best diagnosis for this subject. This is an example where the patient can be diagnosed as suffering from a mixed dementia, as the diagnosis obtained through via the method of the present invention slightly differs, but nevertheless differs from the diagnosis obtained through conventional clinical methods.

For the other subjects, which were presenting a low level of noradrenaline concentration (<3000 pmol/l), the mathematical processing of the clinical data has allowed for the refinement of global note in relation with AD2.

For instance, for subject 13, the initial clinical diagnosis has concluded that the subject was suffering from VD. This was first confirmed with the mathematical algorithm that does not take into account the presence of arterial hypertension because the global note (without taking into account the arterial hypertension) was the highest for DLB (22.47). However, in reason of an amount of noradrenaline concentration in the blood (2470 pmol/l) inferior to a predetermined threshold (e.g. inferior to 3000 pmol/l), the subject has been suspected to suffer from AD2. The arterial hypertension was thus taken into account and has allowed for the refinement of the diagnosis. The subject 13 is 79 years old (thus □=3) and shows symptoms of arterial hypertension (thus □=]0.0; 1.0] (0.0 being excluded, due the presence of symptoms of arterial hypertension), in this case, □=0.10). With this refinement, the global note for AD2 has increased to 22.59 and has become the highest calculated global note, which means that the subject 13 can be effectively considered as suffering from AD2.

The following data of sensitivity and specificity in relation with each screened disease are given. It is highlighted that an accurate diagnosis is characterized by a combination of sensitivity and specificity amounting to at most 200. Those data are given in Table V.

TABLE V
Performance of the diagnosis method of the present invention.
Combination means the sum of sensitivity and specificity

| | | | | | Without | By |
|---|---|---|---|---|---|---|
| | | | | | using | using |
| | Without using V^{HT} | | By using V^{HT} | | V^{HT} | V^{HT} |
| | Sensitivity | Specificity | Sensitivity | Specificity | Combination | |
| AD2 | 81.48 | 83.56 | 85.19 | 83.56 | 165.04 | 168.75 |
| VD | 14.29 | 96.77 | 14.29 | 97.84 | 111.06 | 112.13 |
| DLB | 87.5 | 94.56 | 87.5 | 94.57 | 182.06 | 182.07 |
| FTD | 60 | 97.78 | 60 | 97.78 | 157.78 | 157.78 |
| AD1 | 100 | 92.21 | 100 | 92.21 | 192.21 | 192.21 |
| PSY | 66.67 | 97.87 | 66.67 | 97.87 | 164.54 | 164.54 |
| NC | 36.84 | 100 | 36.84 | 100 | 136.84 | 136.84 |

Table V shows that the inclusion of the parameter of the arterial hypertension status allows to increase the accuracy of the diagnosis, notably in the detection of AD2, VD, and DLB. The diagnosis regarding AD1, PSY, FTD and NC are equivalent to the results obtained without the additional criterion of the arterial hypertension status.

It means that when a subject is presenting a noradrenaline concentration that is lower or equal to a predetermined threshold, preferentially lower or equal to 3000 pmol/l, than the diagnosis method of the invention allows for a better accuracy in certain diseases (AD2 but also VD, and DLB), which in global, render the diagnosis test more precise and allows for the clinician to adjust the treatment in consequence.

The results in term of performance can be explained as followed: the inclusion of the arterial hypertension parameter allows for declassifying of a subject wrongly classified in a certain group of disease which has for effect to increase the specificity of the diagnostic method with regard to this specific disease. As shown in the example of subject 13, a wrongly-VD-diagnosed patient has been declassified from the VD group. In the same time, with regard to AD2, the sensitivity of the diagnostic method increases since the inclusion of the arterial hypertension parameter is an effect to reclassify a wrongly-diagnosed patient into this specific group of AD2. In the example of subject 13, the wrongly-VD-diagnosed patient has been reclassified as AD2 patient.

### Test and Determination Methods

Blood collected in lithium heparin tubes was centrifuged at 3500 rpm for 10 min at +4° C. 1 mL plasma and 100 μL of 2,3-dihydroxybenzoic acid (DHBA) internal standard (from 100 nM DHBA stock solution) was added to extraction tubes with 100 mg of aluminum oxide previously activated with a mixture of 500 μL TRIS 3M and 100 μL EDTA 10% (5:1) according to a known method (Anton A. H., et al., J. Pharmacol. Exp. Ther., 1962, 138(3), 360-375). Samples were stirred for 10 min and centrifuged at 2000 rpm at 4° C. The aluminum oxide with bound catecholamines was then washed 3 times with 5 mL of distilled water followed by centrifugation. The elution of catecholamines was achieved by the addition of 500 μL of HCIO₄ 0.2 N and subsequent centrifugation at 2000 rpm for 10 min at 4° C. Aliquots of 100 μL were injected into the HPLC system (Waters 515 HPLC Pump, Waters Model 717 autosampler injector), which was equipped with a Purospher® STAR RP-C18 endcapped (5 μm). The mobile phase consisted of 50 mM sodium acetate buffer containing 0.9 mM sodium lauryl sulfate, 0.3 mM EDTA, 17.5 mM acetic acid and 12% methanol (vol/vol) at pH 3.6. Electrochemical detection (Coulochem II detector) was performed using an ESA 5010 cell with a glassy carbon working electrode set at a potential of +360 mV vs. Ag/AgCl.

The catecholamines (adrenaline, noradrenaline and dopamine) are extracted from the plasma matrix by adsorption on alumina before the HPLC analysis. Sample preparation is simple, because pH-adjustment of the plasma samples is not necessary. Moreover, samples preparation requires only washing steps with Wash Buffer. A selected HPLC column in combination with mobile phase, optimised for this particular separation, allows for sure and reliable chromatographic quantification. With a certified HPLC kit, one person can analyse up to 100 plasma samples per day.

Extraction: Label a sample clean up cartridge appropriately for each sample. Add 0.5 ml extraction buffer to each cartridge and shake briefly. Then add 1 ml plasma (total capacity of the cartridge is 1.5 ml) and 50 μl Internal Standard (=600 pg DHBA). Close the cartridge with the top plug and mix for 10 min. Then remove the bottom plug from the cartridge and remove the plasma supernatant by using a vacuum equipment or centrifugation (place the cartridge in a disposable centrifugation tube and centrifuge 1 min at 2000 rpm).

Wash steps: Re-mount the bottom plug and remove the top plug. Add 1 ml Wash Buffer. Close the cartridge again and mix for 30 s (vortex). Then remove the bottom plug from the cartridge and remove the plasma supernatant by using a vacuum equipment or centrifugation (place the cartridge in a disposable centrifugation tube and centrifuge 1 min at 2000 rpm). Repeat this step 2 more times. After the last (third) wash step dry the cartridges well by centrifugation (2 min at 4000 rpm). To ensure that the Wash Buffer is removed completely, tap at the cartridge; the alumina should loosen from the frit. Discard bottom plug.

1. An in vitro method for performing a differential diagnosis of neurodegenerative diseases selected from early stage of Alzheimer's disease (AD1), advanced stage of Alzheimer's disease (AD2), mental depression (PSY), dementia with Lewy bodies (DLB), frontotemporal dementia (FTD), and/or vascular dementia (VD); said method being performed in a subject presenting signs of dementia;
said method comprising the steps of:
a) determining at least five criteria of said subject, said at least five criteria comprising the age of said subject, a score of said subject to a questionnaire adapted for screening cognitive function, a dopamine concentration [D] in a blood sample of said subject, an adrenaline concentration [A] in said blood sample of said subject and a noradrenaline concentration [NA] in said blood sample of said subject,
c) calculating one global note per each neurodegenerative disease according to the following sub-steps:
i) determining for one neurodegenerative disease one parameter V_{disease}^{criterion} per criterion, said parameter combining one criterion selected from the five criteria determined in step (a) with reference values of said one criterion in function of said one neurodegenerative disease,
ii) summing each parameter V_{disease}^{criterion} related to said one neurodegenerative disease obtained in sub-step (i) so as to obtain one global note in relation with said one neurodegenerative disease;
iii) repeating sub-steps (i) and (ii) for each neurodegenerative disease,
d) determining, based on each global note per neurodegenerative disease calculated in steps (c), whether said subject suffers from one or more neurodegenerative diseases selected from early stage Alzheimer's disease (AD1), advanced stage of Alzheimer's disease (AD2), mental depression (PSY), dementia with Lewy bodies (DLB), frontotemporal dementia (FTD), and/or vascular dementia (VD);
characterized in that the method further comprises:
a step (b) performed after step (a); wherein step (b) comprises the step of comparing said noradrenaline concentration [NA] to a predetermined threshold, and if said noradrenaline concentration [NA] is lower or equal to said predetermined threshold, then determining the arterial hypertension status of said subject,
and in that step (c) further comprises, if said noradrenaline concentration [NA] is lower or equal to said predetermined threshold, the step of calculating the global note in relation with advanced stage of Alzheimer's disease (AD2) by adding a parameter V\hat{ }HT in relation with the arterial hypertension status of said subject.

2. The method according to claim 1, characterized in that the predetermined threshold of step (b) is a noradrenaline concentration [NA] comprised between 2900 pmol/l and 3100 pmol/l; and/or in that said steps (b), (c) and (d) are carried out by a computer.

3. The method according to claim 1, characterized in that said subject is in homeostatic imbalance.

4. The method according to claim 1, characterized in that said parameter V_{disease}^{criterion} per criterion in function of each neurodegenerative disease is determined in sub-step (i) according to the following mathematical equation (1):
$${V}_{d i s e a s e}^{criterion} = 1 + K \frac{V A {L}^{c r i t e r i o n} - {MINI}_{d i s e a s e}^{c r i t e r i o n}}{{MAXI}_{d i s e a s e}^{c r i t e r i o n} - {MINI}_{d i s e a s e}^{c r i t e r i o n}} \qquad ( 1 )$$
wherein VAL^{criterion} is the measured value of one criterion selected from the five criteria determined in step (a),
wherein MINI_{disease}^{criterion} is the minimum reference value with respect to each neurodegenerative disease in function of said criterion,
wherein MAXI_{disease}^{criterion} is the maximum reference value with respect to each neurodegenerative disease in function of said criterion, and
wherein K is a mathematical parameter, K being comprised between 0.01 and 1.00, preferentially between 0.05 and 0.95.

5. The method according to claim 1, characterized in that it further comprises the step of attributing a weighting factor to each parameter V_{disease}^{criterion} with preference, said weighting factor:
is comprised between 0.1 and 3.0 with respect to the parameter in relation with the score to a questionnaire adapted for screening cognitive function of said subject; and/or
is comprised between 1.0 and 10.0 with respect to one or more parameters selected from the parameter in relation with the age, the parameter in relation with the dopamine concentration [D], the parameter in relation with the adrenaline concentration [A], and the parameter in relation with the noradrenaline concentration [NA].

6. The method according to claim 1, characterized in that said parameter V^{HT} in relation with the arterial hypertension status of said subject is function of the age of said subject, and is comprised between 0 and 11.

7. The method according to claim 1, wherein said parameter V^{HT} in relation with the arterial hypertension status of said subject is determined according to the following mathematical equation (2)
V^{HT}=α(age)+β (2)
wherein α is a variable in function of the age of said subject, with preference α is a value comprised between 0 and 10.0, and
wherein β is a variable in function of the seriousness of the arterial hypertension status, with preference β is a value comprised between 0 and 1.0.

8. The method according to claim 1, wherein the reference values used in step (c) in relation with early stage of Alzheimer's disease (AD1) are as followed:
with respect to the age of said subject, the minimum reference value is at least 50 years and/or the maximum reference value is at most 90 years,
with respect to the score of said subject to a questionnaire adapted for screening cognitive function, the minimum reference value is at least 14 and/or the maximum reference value is at most 29;
with respect to the dopamine concentration [D], the minimum reference value is at least 90 pmol/l, and/or the maximum reference value is at most 1000 pmol/l;
with respect to the adrenaline concentration [A] the minimum reference value is at least 40 pmol/l, and/or the maximum reference value is at most 2000 pmol/l,
with respect to the noradrenaline concentration [NA] the minimum reference value is at least 2900 pmol/l, and/or the maximum reference value is at most 9000 pmol/l.

9. The method according to claim 1, wherein the reference values used in step (c) in relation with advanced stage of Alzheimer's disease (AD2) are as followed:
with respect to the age of said subject, the minimum reference value is at least 45 years and/or the maximum reference value is at most 95 years,
with respect to the score of said subject to a questionnaire adapted for screening cognitive function, the minimum reference value is at least 3 and/or the maximum reference value is at most 30;
with respect to the dopamine concentration [D], the minimum reference value is at least 140 pmol/l, and/or the maximum reference value is at most 1000 pmol/l;
with respect to the adrenaline concentration [A] the minimum reference value is at least 80 pmol/l, and/or the maximum reference value is at most 1500 pmol/l,
with respect to the noradrenaline concentration [NA] the minimum reference value is at least 750 pmol/l, and/or the maximum reference value is at most 3000 pmol/l.

10. The method according to claim 1, wherein the reference values used in step (c) in relation with mental depression (PSY) are as followed:
with respect to the age of said subject, the minimum reference value is at least 35 years and/or the maximum reference value is at most 80 years,
with respect to the score of said subject to a questionnaire adapted for screening cognitive function, the minimum reference value is at least 21 and/or the maximum reference value is at most 30;
with respect to the dopamine concentration [D], the minimum reference value is at least 250 pmol/l, and/or the maximum reference value is at most 750 pmol/l;
with respect to the adrenaline concentration [A] the minimum reference value is at least 180 pmol/l, and/or the maximum reference value is at most 450 pmol/l,
with respect to the noradrenaline concentration [NA] the minimum reference value is at least 1000 pmol/l, and/or the maximum reference value is at most 4000 pmol/l.

11. The method according to claim 1, wherein the reference values used in step (c) in relation with dementia with Lewy bodies (DLB) are as followed:
with respect to the age of said subject, the minimum reference value is at least 55 years and/or the maximum reference value is at most 85 years,
with respect to the score of said subject to a questionnaire adapted for screening cognitive function, the minimum reference value is at least 7 and/or the maximum reference value is at most 25;
with respect to the dopamine concentration [D], the minimum reference value is at least 150 pmol/l, and/or the maximum reference value is at most 400 pmol/l;
with respect to the adrenaline concentration [A] the minimum reference value is at least 80 pmol/l, and/or the maximum reference value is at most 700 pmol/l;
with respect to the noradrenaline concentration [NA] the minimum reference value is at least 900 pmol/l, and/or the maximum reference value is at most 8000 pmol/l.

12. The method according to claim 1, wherein the reference values used in step (c) in relation with frontotemporal dementia (FTD) are as followed:
with respect to the age of said subject, the minimum reference value is at least 45 years and/or the maximum reference value is at most 80 years,
with respect to the score of said subject to a questionnaire adapted for screening cognitive function, the minimum reference value is at least 0 and/or the maximum reference value is at most 28;
with respect to the dopamine concentration [D], the minimum reference value is at least 200 pmol/l, and/or the maximum reference value is at most 1100 pmol/l;
with respect to the adrenaline concentration [A] the minimum reference value is at least 100 pmol/l, and/or the maximum reference value is at most 1500 pmol/l;
with respect to the noradrenaline concentration [NA] the minimum reference value is at least 700 pmol/l, and/or the maximum reference value is at most 6000 pmol/l.

13. The method according to claim 1, wherein the reference values used in step (c) in relation with vascular dementia (VD) are as followed:
with respect to the age of said subject, the minimum reference value is at least 55 years and/or the maximum reference value is at most 90 years,
with respect to the score of said subject to a questionnaire adapted for screening cognitive function, the minimum reference value is at least 20 and/or the maximum reference value is at most 29;
with respect to the dopamine concentration [D], the minimum reference value is at least 140 pmol/l, and/or the maximum reference value is at most 580 pmol/l;
with respect to the adrenaline concentration [A] the minimum reference value is at least 200 pmol/l, and/or the maximum reference value is at most 1800 pmol/l,
with respect to the noradrenaline concentration [NA] the minimum reference value is at least 1400 pmol/l, and/or the maximum reference value is at most 4000 pmol/l.

14. The method according to claim 1, characterized in that, the step (c) further comprises calculating one global note in relation with neurological control (NC); with preference, the reference values used in step (c) in relation with neurological control (NC) are as followed:
with respect to the age of said subject, the minimum reference value is at least 35 years and/or the maximum reference value is at most 90 years,
with respect to the score of said subject to a questionnaire adapted for screening cognitive function, the minimum reference value is at least 22 and/or the maximum reference value is at most 30;
with respect to the dopamine concentration [D], the minimum reference value is at least 180 pmol/l, and/or the maximum reference value is at most 1600 pmol/l;
with respect to the adrenaline concentration [A] the minimum reference value is at least 110 pmol/l, and/or the maximum reference value is at most 2500 pmol/l,
with respect to the noradrenaline concentration [NA] the minimum reference value is at least 900 pmol/l, and/or the maximum reference value is at most 7000 pmol/l.

15. A data processing system comprising a processor configured to perform the method in accordance with claim 1 when said steps (b), (c) and (d) are carried out by a computer.
